(19) United States
(12) Patent Application Publication
(10) Pub. No.: US 2026/0294513 A1
(43) Pub. Date: Oct. 1, 2026

(54) TECHNOLOGIES FOR SPOT COAGULATION CONTROL FOR ENERGY-BASED SURGICAL INSTRUMENTS

(71) Applicant: Cilag GmbH International, Zug (CH)

(72) Inventors: Frederick E. Shelton, IV, Hillsboro, OH (US); Jason L. Harris, Cincinnati, OH (US); Jeffrey D. Messerly, Cincinnati, OH (US)

(21) Appl. No.: 19/344,886

(22) Filed: Sep. 30, 2025

Related U.S. Application Data
(60) Provisional application No. 63/780,382, filed on Mar. 30, 2025.

(51) Int. Cl. A61B 18/14; A61B 18/00

(52) U.S. Cl. CPC A61B 18/1445; A61B 2018/00589; A61B 2018/00702; A61B 2018/00875

(57) ABSTRACT

Surgical instruments and associated methods for preventing tissue ingress into the instrument and for controlling the ultrasonic and RF mode of the surgical instrument include an end effector having a pair assembly and a trigger assembly operable to move the jaw assembly of the end effector between the open state and the closed state. The surgical instrument includes a controller configured to close the jaw assembly to a target gap distance in response to a user request for spot coagulation mode, determine whether one or more spot coagulation mode preconditions are satisfied in response to closing the jaw assembly, and activate delivery of spot coagulation energy to the jaw assembly if the one or more preconditions are satisfied.

### CROSS-REFERENCE TO RELATED APPLICATION

This application claims the benefit of and priority to U.S. Patent Application No. 63/780,382, entitled “TECHNOLOGIES FOR TRACKING TISSUE PAD HEIGHT AND ADJUSTING HARMONIC ALGORITHMS BASED ON TISSUE PAD HEIGHT,” which was filed on Mar. 30, 2025, and which is incorporated herein by reference in its entirety.

### TECHNICAL FIELD

The present disclosure relates generally to energy-based surgical instruments and, more particularly, to ultrasonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient’s tissue to create a hemostatic seal. In electro-surgical instruments that do not include an ultrasonic mode (i.e., do not include an ultrasonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including an ultrasonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient’s tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, an energy-based surgical instrument includes an end effector, a mode selector, and a controller. The end effector has a jaw assembly moveable between an open state and a closed state. The jaw assembly includes a jaw clamp with a plurality of electrodes. The mode selector is operable by a user of the surgical instrument. The controller is configured to determine whether the user has requested spot coagulation mode with the mode selector, to close the jaw assembly to a target gap distance in response to a determination that the user has requested the spot coagulation mode, to determine whether one or more spot coagulation mode preconditions are satisfied in response to a closing of the jaw assembly, and to activate delivery of spot coagulation energy to the jaw assembly in response to a determination that the one or more spot coagulation mode preconditions are satisfied.

In some embodiments, the controller is further configured to adjust the target gap distance based on one or more sensed parameters. In some embodiments, the one or more sensed parameters include tissue impedance, tissue conductivity, tissue type, sensed tissue location along the jaw assembly, selective electrode utilization, applied tissue pressure, or initial jaw aperture. In some embodiments, the controller is further configured to adjust the target gap distance based on one or more parameters determined by a smart visualization system, wherein the one or more parameters comprise tissue thickness, tissue type, or bleeder size. In some embodiments, the controller is further configured to adjust the target gap distance based on one or more parameters determined by tissue impedance spectroscopy.

In some embodiments, to close the jaw assembly includes to control a rate of closure of the jaw assembly to a slow rate. In some embodiments, to close the jaw assembly includes to determine whether an impedance between electrodes of the jaw assembly indicates that tissue is contacting the jaw assembly, and to stop closure of the jaw assembly in response to a determination that the impedance indicates that tissue is contacting the jaw assembly.

In some embodiments, to determine whether the one or more spot coagulation mode preconditions are satisfied includes to determine whether the jaw assembly has reached the target gap distance. In some embodiments, to determine whether the one or more spot coagulation mode preconditions are satisfied includes to compare a conductance between electrodes of the jaw assembly to a predetermined threshold conductance. In some embodiments, to determine whether the one or more spot coagulation mode preconditions are satisfied includes to compare a pressure from an end effector of the surgical instrument to tissue to a predetermined threshold pressure. In some embodiments, to determine whether the one or more spot coagulation mode preconditions are satisfied includes to determine that electrodes of the jaw assembly are not contacting a metallic object.

In some embodiments, the controller is further configured to determine a first power level for the spot coagulation energy based on one or more sensed parameters. To activate the delivery of the spot coagulation energy includes to activate the delivery of the spot coagulation energy at the first power level. In some embodiments, the controller is further configured to control a jaw gap of the jaw assembly while spot coagulation energy is delivered to the jaw assembly.

According to yet another aspect of the present disclosure, a method for controlling an energy-based surgical instrument includes determining, by a controller of the surgical instrument, whether a user has requested spot coagulation mode; closing, by the controller, a jaw assembly of the surgical instrument to a target gap distance in response to determining that the user has requested the spot coagulation mode; determining, by the controller, whether one or more spot coagulation mode preconditions are satisfied in response to closing the jaw assembly; and activating, by the controller, delivery of spot coagulation energy to the jaw assembly in response to determining that the one or more spot coagulation mode preconditions are satisfied.

In some embodiments, the method further includes adjusting, by the controller, the target gap distance based on one or more sensed parameters. In some embodiments, the one or more sensed parameters include tissue impedance, tissue conductivity, tissue type, sensed tissue location along the jaw assembly, selective electrode utilization, applied tissue pressure, or initial jaw aperture. In some embodiments, the method further includes adjusting, by the controller, the target gap distance based on one or more parameters determined by a smart visualization system, wherein the one or more parameters comprise tissue thickness, tissue type, or bleeder size. In some embodiments, the method further includes adjusting, by the controller, the target gap distance based on one or more parameters determined by tissue impedance spectroscopy.

In some embodiments, closing the jaw assembly includes controlling a rate of closure of the jaw assembly to a slow rate. In some embodiments, closing the jaw assembly includes determining whether an impedance between electrodes of the jaw assembly indicates that tissue is contacting the jaw assembly; and stopping closure of the jaw assembly in response to determining that the impedance indicates that tissue is contacting the jaw assembly.

In some embodiments, determining whether the one or more spot coagulation mode preconditions are satisfied includes determining whether the jaw assembly has reached the target gap distance. In some embodiments, determining whether the one or more spot coagulation mode preconditions are satisfied includes comparing a conductance between electrodes of the jaw assembly to a predetermined threshold conductance. In some embodiments, determining whether the one or more spot coagulation mode preconditions are satisfied includes comparing a pressure from an end effector of the surgical instrument to tissue to a predetermined threshold pressure. In some embodiments, determining whether the one or more spot coagulation mode preconditions are satisfied includes determining that electrodes of the jaw assembly are not contacting a metallic object.

In some embodiments, the method further includes determining, by the controller, a first power level for the spot coagulation energy based on one or more sensed parameters; wherein activating the delivery of the spot coagulation energy comprises activating the delivery of the spot coagulation energy at the first power level. In some embodiments, the method further includes controlling, by the controller, a jaw gap of the jaw assembly while spot coagulation energy is delivered to the jaw assembly.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8A is a perspective view of an embodiment of the jaw clamp assembly used for external spot coagulation;

FIG. 8B is a perspective view of the embodiment of the jaw clamp assembly of FIG. 8A with an adjustable jaw gap;

FIG. 8C is a perspective view of the embodiment of the jaw clamp assembly of FIG. 8A with the jaw gap adjusted for external spot coagulation; and

FIG. 9 is a simplified flow diagram of an embodiment of a method for utilizing the jaw assembly of FIGS. 8A-8C for spot coagulation, which may be executed by the control circuit of FIG. 7.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient’s natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

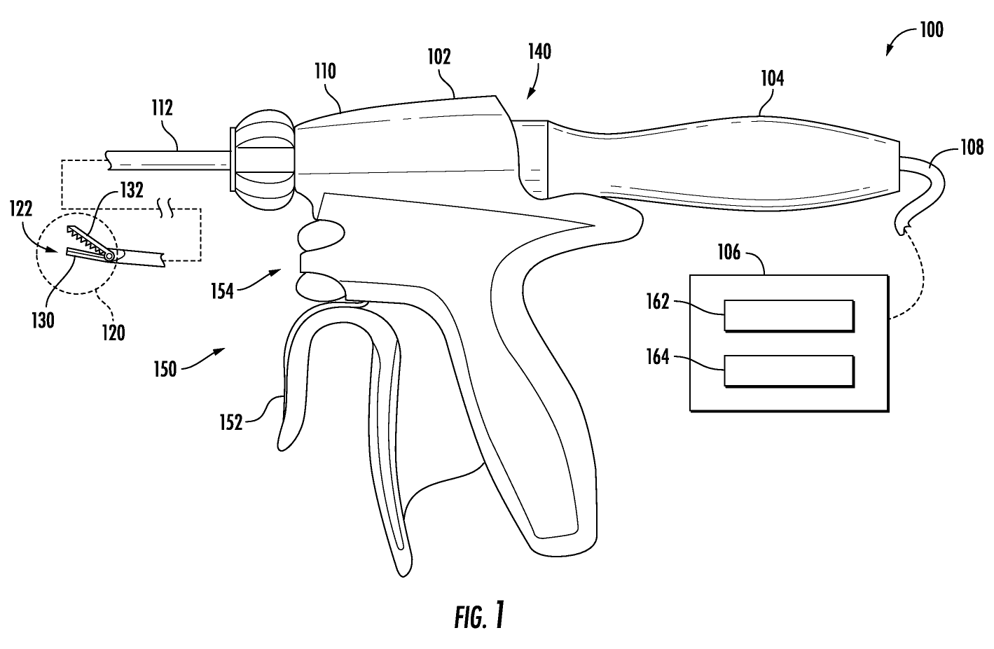

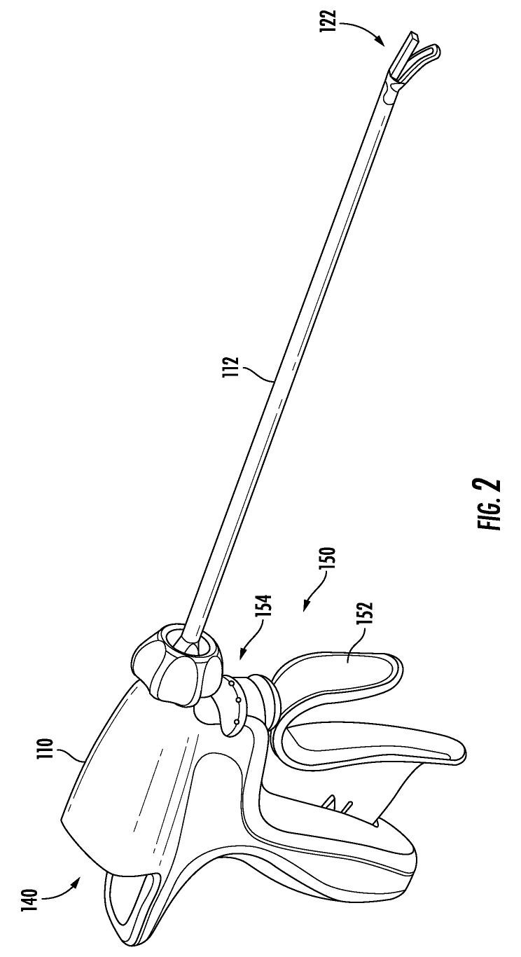

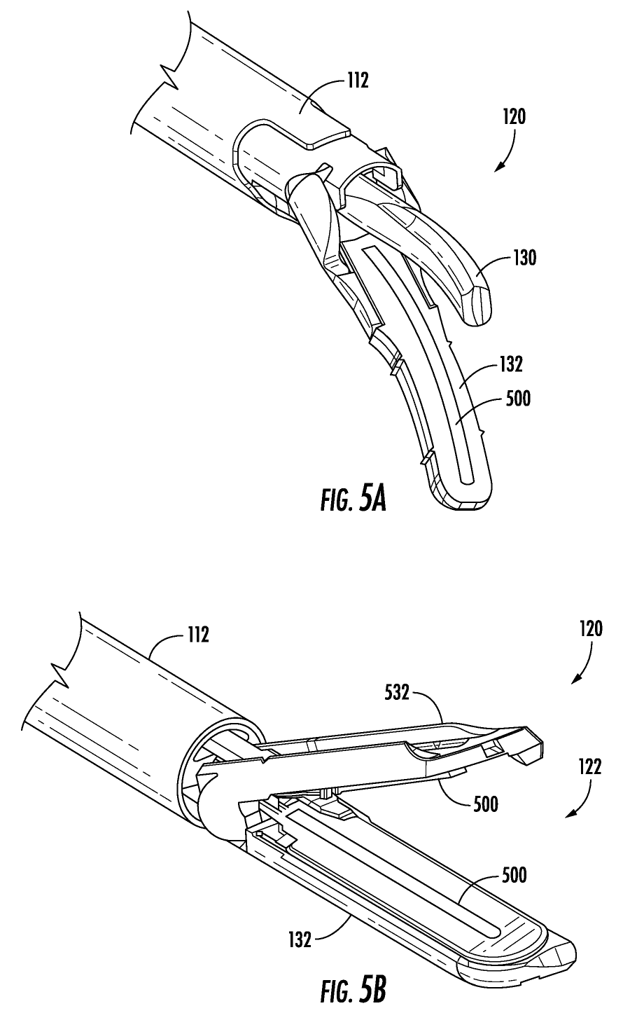

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

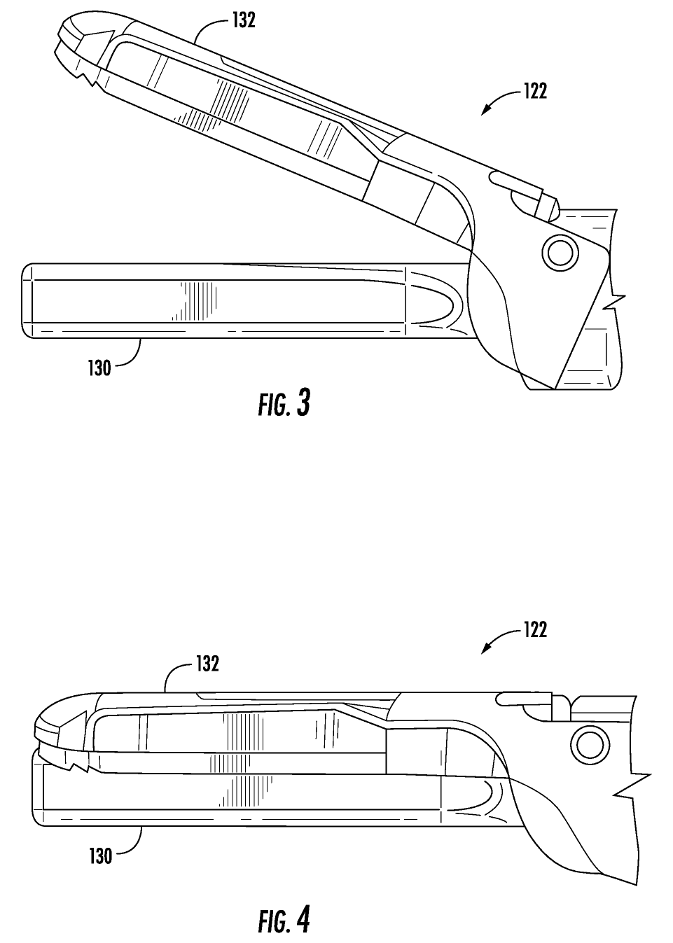

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient’s body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient’s tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient’s tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient’s tissue. For example, the electro-surgical generator module 164 may be configured to measure an impedance of the patient’s tissue using the electrodes 500 and a suitable RF energy level.

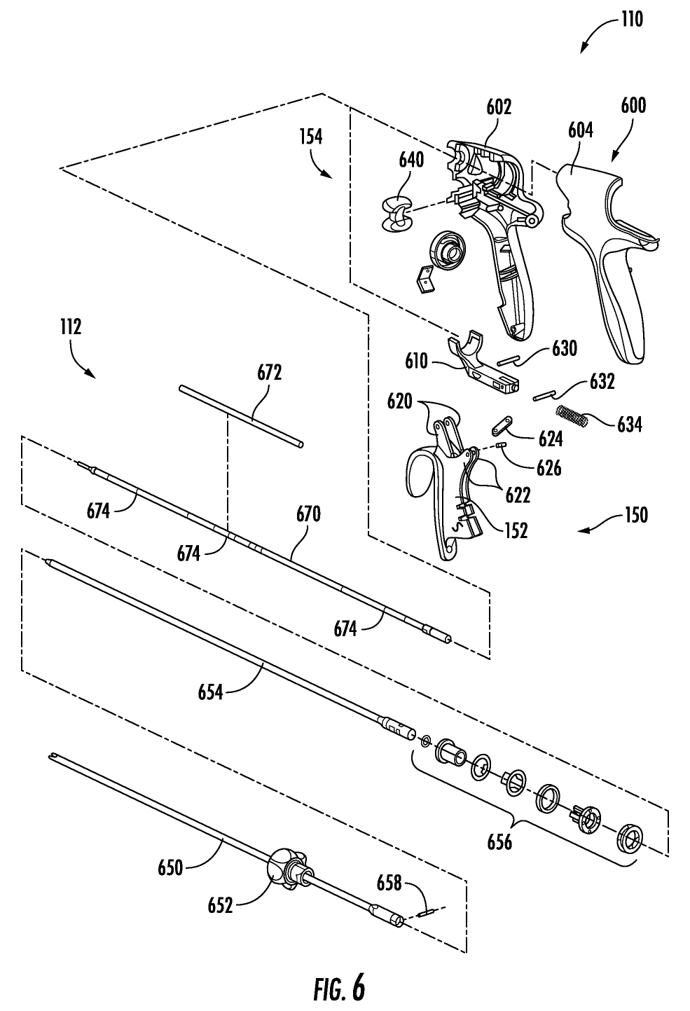

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

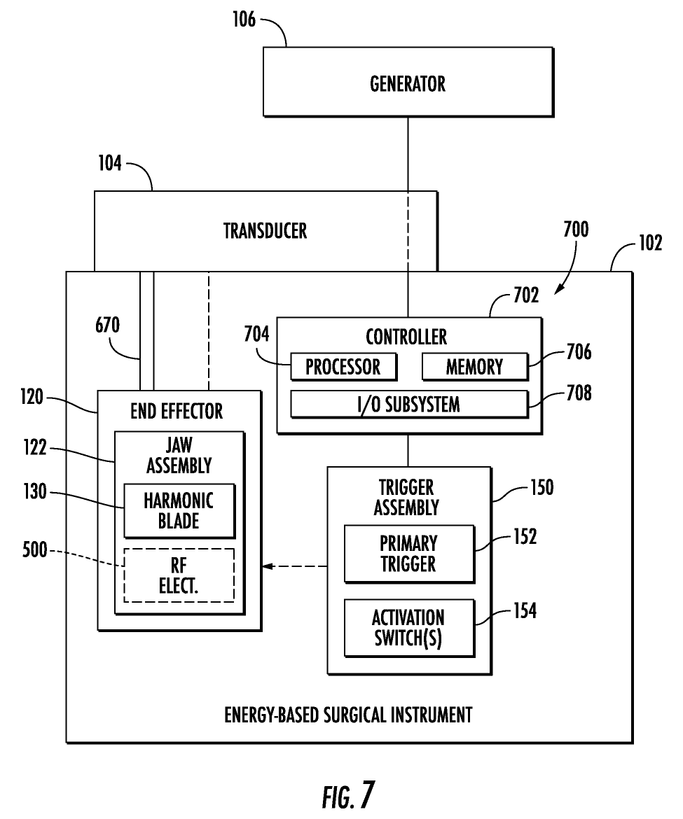

Referring now to FIG. 7, in the illustrative embodiment, the surgical instrument 102 includes a control circuit 700. The control circuit 700 includes a controller 702 and the trigger assembly 150, which cooperate to provide ultrasonic energy to the ultrasonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the ultrasonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

Tissue accumulation around the electrodes 500 in the jaw clamp 132 can diminish the performance and/or efficiency of the surgical instruments 102. In some embodiments, the shape of the electrode 500 may be changed to prevent or reduce tissue accumulation when the surgical instrument 102 is used. In other embodiments, the shape of the jaw clamp 132 may be changed or the jaw clamp 132 may be coupled with an attachment to prevent or reduce tissue accumulation around the electrodes 500.

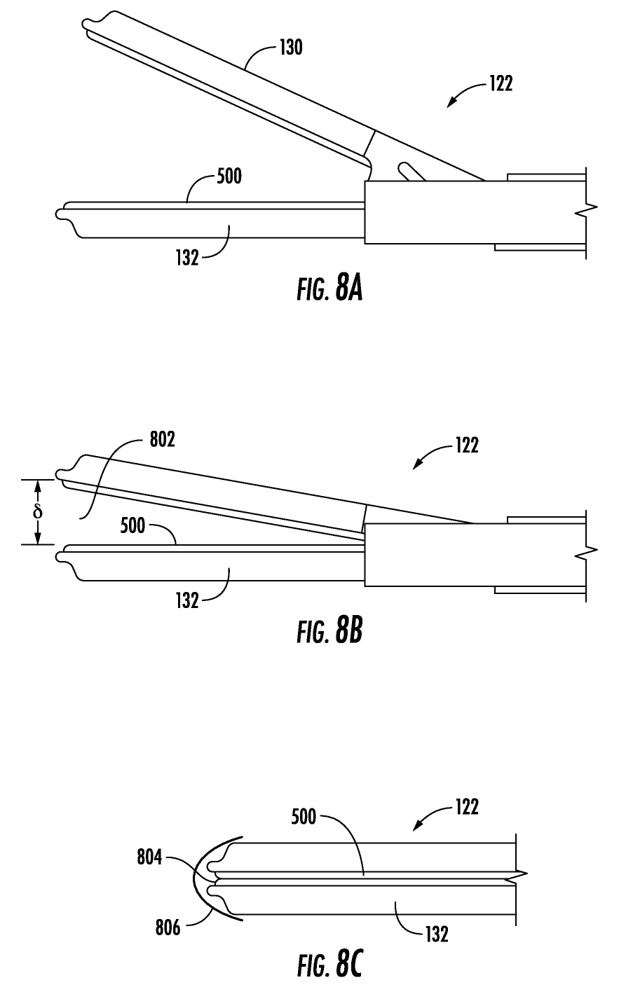

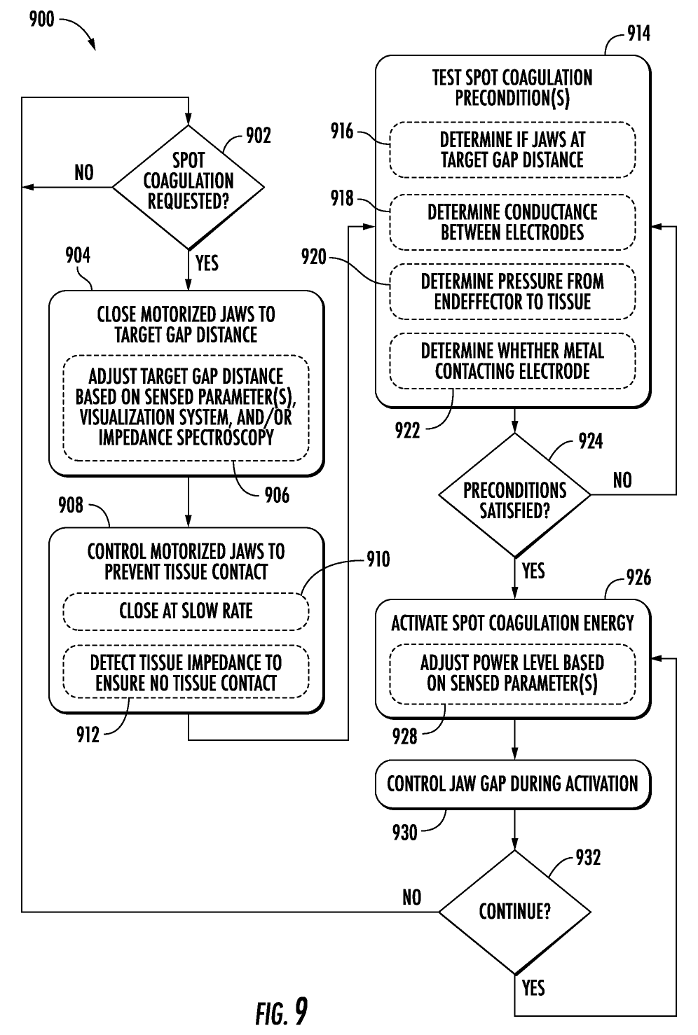

Referring now to FIGS. 8A-8C, in the illustrative embodiment, the jaw assembly 122 of the end effector 120 of the surgical instrument 102 can be used for external spot coagulation. Automated adjustment of a jaw gap 802 between the jaws (e.g., the jaw clamp 132 and the blade 130) illustrated in FIG. 8B for spot coagulation external to the jaw clamp 132 is effected when the user indicates a need for spot coagulation, for example using one or more buttons of the switch assembly 154 or other controls of the surgical instrument 102. Once the need for external spot coagulation is determined, the jaw clamp 132 and the blade 130 are positioned at a predefined jaw gap 804 close to tissue 806, as illustrated in in FIG. 8C. This jaw gap 804 is based on the energy settings of the system 100, the energy modality selected by the user, and the tissue parameters sensed or known by the system 100. When certain preconditions are satisfied, bipolar energy is used to couple the outside facing aspect of the first jaw clamp 132 to the second jaw (e.g., the blade 130) with the tissue coupled in-between to provide an externally focused energy coagulation. The preconditions include but are not limited to the presence of conductive electrodes 500 between the jaw clamp 132 and the blade 130, the presence of tissue between the jaw clamps 132 and the blade 130, an adequate pressure applied by the end effector 120 to the tissue, and the absence of any metal connecting the electrode 500 (for example, a clip or staple present within the jaws). External spot coagulation requires active adaptation of the jaw assembly 122 in order to position the electrodes 500 into an orientation able to utilize bipolar energy to effect externalized tissue welding by the jaw clamp 132 and the blade 130.

Referring now to FIG. 9, the controller 702 of the energy-based surgical instrument 102 may be configured to execute a method 900 for effecting external spot coagulation. The method 900 begins with block 902, in which the controller 702 determines whether a user indicates the need for spot coagulation. The user may indicate the need for spot coagulation, for example, by activating one or more buttons of the switch assembly 154, a trigger mechanism, an energy mode selector, or other user controls of the surgical instrument 102. If the controller 702 determines that the user has not indicated a need for spot coagulation, the method 900 loops back to block 902 to continue monitoring for a user indication. If the controller 702 determines that the user has indicated a need for spot coagulation, the method 900 proceeds to block 904.

In block 904, the controller 702 causes motorized jaws (e.g., the jaw clamp 132 and the blade 130) of the jaw assembly 122 of the end effector 120 to close to a target gap distance 804. In some embodiments, the jaws 132, 130 can be opened and closed based on user preference by activating the trigger assembly 150. As an illustrative example, if there is no tissue between the jaws 132, 130 and no load is measured by the controller 702, indicating the jaws 132, 130 are empty (e.g., no motor load, no motor current, or other indicator that the jaws 132, 130 are empty), when the user indicates the need for spot coagulation, the jaws 132, 130 close to the target gap distance 804, which may be adjusted based on one or more variables or other parameters measured by the controller 702.

In some embodiments, in block 906 the controller 702 adjusts the target gap distance 804 based on one or more sensed parameters(s), such as tissue impedance, tissue conductivity, tissue type, sensed tissue location along the jaw assembly, selective electrode utilization, applied tissue pressure, or initial jaw aperture. The jaw gap may be determined based on feathering of the tissue with a solid organ, bleeder size, skeletonization, and tissue separation. In some embodiments, for example if the system 100 includes a smart generator 106 which has cooperative interaction with a visualization system, the visualization system may be used to determine the tissue thickness, tissue type, or bleeder size, which may be used to adjust the target jaw gap distance 804. In some embodiments, tissue impedance spectroscopy (i.e., utilizing a range of frequencies, rather than just one frequency, to interrogate the tissue in the sending mode) may be used to adjust the target jaw gap distance 804, for example when exterior visualization or external data may not be utilized to determine tissue type or bleeder magnitude.

In block 908, the controller 702 controls closure of the motorized jaws 132, 130 to prevent tissue contact or otherwise prevent tissue trauma. For example, in some embodiments the controller 702 may control motor torque, motor current, or otherwise control motor deployment to limit the closing force exerted by the jaws 132, 130. Continuing that example, in some embodiments, the force applied may range from between about 5 lbs. to about 10 lbs. axial load, including any force or range comprised therein. As another example, the motor current applied may range from about 0.1 A to about 0.5 A including any current or range comprised therein. In some embodiments, in block 910 the controller 702 may control the rate of movement of the jaws 132, 130 to be limited to a slow rate. For example, the jaws 132, 130 may take about 1 second to 2 seconds to be fully positioned at the target jaw gap distance 804.

In some embodiments, in block 912 the controller 702 detects tissue impedance between electrodes 500 of the jaw assembly 122 to ensure there is no tissue contact. For example, the controller 702 may determine whether impedance between the electrodes 500 is within a predetermined range of impedance values associated with tissue (e.g., a range of intermediate impedance values, not open-circuit and not short-circuit, etc.), and if the impedance is within that range, stop closure of the jaws 132, 130.

In block 914, the controller 702 tests or otherwise evaluates one or more preconditions for performing spot coagulation. Preconditions for spot coagulation may include one or more sensed parameters, system states, or other conditions that must be satisfied before the surgical instrument 102 activates energy for spot coagulation. In some embodiments, in block 916 the controller 702 determines whether the jaws 132, 130 of the jaw assembly 122 have reached the target jaw gap distance 804. In some embodiments, in block 918 the controller 702 determines conductance between the electrodes 500. For example, the controller 702 may determine whether the conductivity (or a related measurement such as resistivity, resistance, impedance, or the like) between the jaws 132, 130 indicates that tissue is contacted by both electrodes 500. The controller 702 may further determine conductivity based on the magnitude of the electrode contact by assessing surface area of the electrodes 500 and the impedance at therapeutic frequency. In some embodiments, in block 920 the controller 702 determines whether adequate pressure from the end-effector 102 to the tissue is applied, for example by comparing a measured pressure to a threshold pressure. In some embodiments, in block 922 the controller 702 determines that there is no sensing of a metal object connecting the electrodes 500 (e.g., a clip or staple present within the jaws). For example, the controller 702 may determine whether a short-circuit condition exists between the electrodes 500. In some embodiments the controller 702 may evaluate one or more combinations of those preconditions and/or may evaluate one or more additional preconditions.

The method 900 proceeds to block 924, in which the controller 702 determines whether all preconditions are satisfied. If any of the preconditions are not satisfied, the method 900 loops back to block 914 to continue testing preconditions. In this circumstance, the surgical instrument 102 does not activate energy for spot coagulation. The controller 702 may continue to monitor for satisfying preconditions until all preconditions are met or until the spot coagulation operation is canceled (e.g., if the user cancels the request by releasing a button of the switch assembly 154 or other interface, if a timeout expires, or another reason). Referring again to block 924, if all of the preconditions are satisfied, the method 900 proceeds to block 926.

In block 926, the controller 702 activates the generator 106 to apply the energy (e.g., RF energy) required for spot coagulation as indicated by the user. In some embodiments, the system 100 may include more than one generator 106. For example, one of the generators 106 may be used for modulating ultrasonic energy and the other may be used for modulating RF energy. In such embodiments, the controller 702 may activate the generator 106 for modulating RF energy to activate the spot coagulation mode. After the jaws 132, 130 are positioned, there may be a minimum impedance trigger, for example as one of the preconditions for spot coagulation as described above. Accordingly, if a minimum impedance is detected, the system 100 may automatically activate the energy for the spot coagulation, allowing the user to just touch the end effector 120 to the intended area in block 926. The detection of minimum impedance would ensure that tissue contact occurs before the jaws are energized. Once the minimum impedance is removed, the controller 702 immediately de-energizes the jaws 132, 130.

In some embodiments, in block 928 the controller 702 adjusts the power level of the coagulation energy (e.g., voltage level, duration of the cycle, or other contributor to power level of coagulation energy) based on the jaw gap determined in block 904 and/or one or more sensed parameters, such as those parameters measured by the controller 702 as described above in connection with block 906. For example, the controller 702 may adjust power level based on tissue impedance, tissue conductivity, tissue type, sensed tissue location along the jaw assembly, selective electrode utilization, applied tissue pressure, initial jaw aperture, or other sensed parameters. In some embodiments, a visualization system may be used to determine the tissue thickness, tissue type, or bleeder size, which may be used to adjust the power level. In some embodiments, tissue impedance spectroscopy (i.e., utilizing a range of frequencies, rather than just one frequency, to interrogate the tissue in the sending mode) may be used to adjust the power level, for example when exterior visualization or external data may not be utilized to determine tissue type or bleeder magnitude. Continuing that example, a spectral impendence fingerprint may be used to define the tissue type (e.g., connective tissue, smooth muscle, fat, adventitia, etc.). In some embodiments, the controller 702 may continue to adjust power level based on sensed parameters or other factors during delivery of the spot coagulation energy.

Similarly, in block 930 the controller 702 may further adjust the jaw gap 802 during progression through the coagulation cycle. For example, the magnitude of the surrounding fluid may affect the power pathway, parasitic power leaching, and the dissipation of the heat that is intended to cause coagulation. The controller 702 may assess the surrounding tissue and the magnitude of the bleed, and cause the system 100 to adapt the power delivery in order to compensate for any change. The controller 702 may further consider the power level of the system 100 and the time of coagulation to determine the jaw gap and the power used for coagulation.

In block 932, the controller 702 determines whether to continue applying spot coagulation energy. For example, in some embodiments, the controller 702 may continue applying spot coagulation energy only while all preconditions continue to be satisfied (e.g., the user has selected spot coagulation mode, for example by pressing a button of the switch assembly 154, the jaws are closed at the target jaw gap distance 804, the end effector 120 is in contact with tissue with sufficient pressure, no metal object is contacting the electrodes, etc.). As another example, in some embodiments the controller 702 may continue applying spot coagulation energy for a predetermined length of time or otherwise for a predetermined coagulation program. As described above, in some embodiments, the controller 702 may change the parameters for spot coagulation (e.g., target jaw gap distance 804, power level, or other parameters) based on the need for maintenance of constant power, constant energy density, and/or constant location of the electrodes on the jaws 132, 130. If the controller 702 determines to continue applying energy, the method 900 loops back to block 926. If the controller 702 determines to stop applying energy, the controller 702 and/or the generator 106 stops delivering the spot coagulation energy to the electrodes 500. The method 900 loops back to block 902, in which the controller 702 monitors for further activation of the spot coagulation mode.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There is a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for controlling an energy-based surgical instrument, the method comprising:
determining, by a controller of the surgical instrument, whether a user has requested spot coagulation mode;
closing, by the controller, a jaw assembly of the surgical instrument to a target gap distance in response to determining that the user has requested the spot coagulation mode;
determining, by the controller, whether one or more spot coagulation mode preconditions are satisfied in response to closing the jaw assembly; and
activating, by the controller, delivery of spot coagulation energy to the jaw assembly in response to determining that the one or more spot coagulation mode preconditions are satisfied.

2. The method of claim 1, further comprising adjusting, by the controller, the target gap distance based on one or more sensed parameters.

3. The method of claim 2, wherein the one or more sensed parameters comprises tissue impedance, tissue conductivity, tissue type, sensed tissue location along the jaw assembly, selective electrode utilization, applied tissue pressure, or initial jaw aperture.

4. The method of claim 1, further comprising adjusting, by the controller, the target gap distance based on one or more parameters determined by a smart visualization system, wherein the one or more parameters comprise tissue thickness, tissue type, or bleeder size.

5. The method of claim 1, further comprising adjusting, by the controller, the target gap distance based on one or more parameters determined by tissue impedance spectroscopy.

6. The method of claim 1, wherein closing the jaw assembly comprises controlling a rate of closure of the jaw assembly to a slow rate.

7. The method of claim 1, wherein closing the jaw assembly comprises:
determining whether an impedance between electrodes of the jaw assembly indicates that tissue is contacting the jaw assembly; and
stopping closure of the jaw assembly in response to determining that the impedance indicates that tissue is contacting the jaw assembly.

8. The method of claim 1, wherein determining whether the one or more spot coagulation mode preconditions are satisfied comprises determining whether the jaw assembly has reached the target gap distance.

9. The method of claim 1, wherein determining whether the one or more spot coagulation mode preconditions are satisfied comprises comparing a conductance between electrodes of the jaw assembly to a predetermined threshold conductance.

10. The method of claim 1, wherein determining whether the one or more spot coagulation mode preconditions are satisfied comprises comparing a pressure from an end effector of the surgical instrument to tissue to a predetermined threshold pressure.

11. The method of claim 1, wherein determining whether the one or more spot coagulation mode preconditions are satisfied comprises determining that electrodes of the jaw assembly are not contacting a metallic object.

12. The method of claim 1, further comprising:
determining, by the controller, a first power level for the spot coagulation energy based on one or more sensed parameters;
wherein activating the delivery of the spot coagulation energy comprises activating the delivery of the spot coagulation energy at the first power level.

13. The method of claim 1, further comprising controlling, by the controller, a jaw gap of the jaw assembly while spot coagulation energy is delivered to the jaw assembly.

14. An energy-based surgical instrument comprising:
an end effector having a jaw assembly moveable between an open state and a closed state, wherein the jaw assembly includes a jaw clamp with a plurality of electrodes;
a mode selector operable by a user of the surgical instrument; and
a controller configured to:
determine whether the user has requested spot coagulation mode with the mode selector;
close the jaw assembly to a target gap distance in response to a determination that the user has requested the spot coagulation mode;
determine whether one or more spot coagulation mode preconditions are satisfied in response to a closing of the jaw assembly; and
activate delivery of spot coagulation energy to the jaw assembly in response to a determination that the one or more spot coagulation mode preconditions are satisfied.

15. The surgical instrument of claim 14, wherein the controller is further configured to adjust the target gap distance based on one or more sensed parameters, wherein the one or more sensed parameters comprises tissue impedance, tissue conductivity, tissue type, sensed tissue location along the jaw assembly, selective electrode utilization, applied tissue pressure, or initial jaw aperture.

16. The surgical instrument of claim 14, wherein to close the jaw assembly comprises to:
determine whether an impedance between electrodes of the jaw assembly indicates that tissue is contacting the jaw assembly; and
stop closure of the jaw assembly in response to a determination that the impedance indicates that tissue is contacting the jaw assembly.

17. The surgical instrument of claim 14, wherein to determine whether the one or more spot coagulation mode preconditions are satisfied comprises to determine whether the jaw assembly has reached the target gap distance, and to compare a conductance between electrodes of the jaw assembly to a predetermined threshold conductance.

18. The surgical instrument of claim 14, wherein to determine whether the one or more spot coagulation mode preconditions are satisfied comprises to compare a pressure from an end effector of the surgical instrument to tissue to a predetermined threshold pressure.

19. The surgical instrument of claim 14, wherein to determine whether the one or more spot coagulation mode preconditions are satisfied comprises to determine that electrodes of the jaw assembly are not contacting a metallic object.

20. The surgical instrument of claim 14, wherein:
the controller is further configured to determine a first power level for the spot coagulation energy based on one or more sensed parameters; and
to activate the delivery of the spot coagulation energy comprises to activate the delivery of the spot coagulation energy at the first power level.
